(19) United States
(12) Patent Application Publication
(10) Pub. No.: US 2021/0393990 A1
(43) Pub. Date: Dec. 23, 2021

(54) ULTRASOUND THERAPY FOR SYSTEMIC IMMUNE MODULATION

(71) Applicant: Donald Murrill Botta, JR., Maitland, FL (US)

(72) Inventor: Donald Murrill Botta, JR., Maitland, FL (US)

(21) Appl. No.: 17/348,850

(22) Filed: Jun. 16, 2021

Related U.S. Application Data
(60) Provisional application No. 63/040,065, filed on Jun. 17, 2020.

(51) Int. Cl. A61N 7/00

(52) U.S. Cl. CPC A61N 7/00; A61N 2007/0004

(57) ABSTRACT

A method of treating a disease in a subject is provided, including delivering ultrasound energy to a treatment area of the subject, wherein said ultrasound energy inactivates the cytokines in the treatment area or downstream thereof.

### CROSS-REFERENCE TO RELATED APPLICATIONS

This application claims the benefit of U.S. Provisional Application No. 63/040,065 filed Jun. 17, 2020 and incorporated herein by reference in its entirety.

### BACKGROUND

Although the mammalian immune system has elements throughout the body, eighty percent of the active immune system resides in or around the gut. The main communication between the gut immune system and the rest of the body is via the thoracic duct (TD) using the thoracic duct lymph (TDL). The main components of the thoracic duct lymph include fats absorbed in the diet, and lymphatic fluid containing immune messenger molecules called cytokines (CK) and white blood cells (WBC) which direct immune responses. Normally, about eighty percent of TDL enters the circulation through the left thoracic duct at the intersection of the left subclavian vein and the left internal jugular vein, where they form the innominate vein. The remainder enters the circulation through the right thoracic duct near the junction of the right internal jugular vein and the right subclavian vein.

Ligation of the TDL results in rapid utilization and formation of collateral lymphatic vessels to deliver the TDL to the circulation. While ligation of the TD may result in transient gut and/or lower extremity edema, it does not result in significant immunosuppression.

Drainage of the TDL does result in profound immunosuppression. In the early days of liver transplantation a series of patients were found to be lymphocyte depleted by draining the TDL prior to liver transplantation. This resulted in the first successful liver transplants. This technique has also been used for renal transplant immunosuppression. There are a number of references to TDL drainage to prevent complications from severe pancreatitis, with remarkable results. In addition, animal models of peritonitis showed improved outcomes with TDL drainage, even as early as the 1920's. TDL drainage does have some drawbacks. First, it is not universally reproducible, as the thoracic duct can be difficult to locate operatively, and it is very fragile. In addition, TDL drainage can result in profound malnutrition and fluid deficits, unless the fluid is re-infused, or replaced, which can become very cumbersome, and is accompanied by its own set of complications.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 provides a schematic view of an embodiment of an ultrasound transducer.

FIG. 2 provides a view of an ultrasound transducer relative to an anatomy of a subject.

FIG. 3 provides a schematic of an embodiment of a system comprising multiple components.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

While drainage of the TDL or ligation of the thoracic duct can be helpful in modulating the immune system, the inventor herein has identified a much simpler, safer, and more effective method of modulating the immune system. The system and methods described herein involve, in one embodiment, deactivation of TDL and its components by ultrasound delivery to the thoracic duct at its junction with the bloodstream.

Ultrasound has been shown to induce apoptosis (programmed cell death) in white blood cells. At certain wavelengths and energy delivery, it has been discovered herein that ultrasound enhances the function of certain WBCs.

The cytokines have large quaternary (three dimensionally folded) structures that can be disrupted, or broken down, with ultrasound. Cytokines can be either pro- or anti-inflammatory, so the immune system can be negatively or positively modulated, depending on which cytokines are inactivated.

The lymphatic vessels targeted by ultrasound therapy in various systems and methods described herein may be used for immunosuppression for organ transplantations, treatment of autoimmune diseases, immunomodulation in acute inflammatory states like trauma, sepsis, viral or bacterial infections, field application to trauma victims to blunt the systemic inflammatory response, including both blunt and penetrating trauma, both in civilian and military settings, treatment and prevention of adult respiratory distress syndrome (ARDS), treatment and prevention of multisystem organ failure (MSOF), treatment and prevention of cytokine storm(s), treatment or prevention of neonatal necrotizing enterocolitis (NEC), pre- intra, and post-operative application to people who have surgical operations to blunt the systemic inflammatory response, and inactivating white blood cells as they pass the transducer, in non-limiting examples.

The immune disorders described herein include, but are not limited to, multiple sclerosis, ankylosing spondylitis, rheumatoid arthritis, celiac disease, myositis, myasthenia gravis, Addison's disease, lupus, hemolytic anemia, vitiligo, scleroderma, psoriasis, Hashimoto's disease, Addison's disease, Grave's disease, reactive arthritis, Sjogren's syndrome, nephritis, chronic Lyme disease, vasculitis, endocarditis, alopecia areata, urticaria, vasculitis, uveitis, pemphigus, erythema nodusum, dermatitis, eczema, Type 1 Diabetes, temporal arteritis, Crohn's Disease, Behcet's disease, and psoriatic arthritis, chronic or acute allergies, atopic forms of bronchial asthma, anaphylaxis, autoimmune hemolytic anemia, autoimmune thrombocytopenic purpura, pemphigus vulgaris, vasculitis caused by antineutrophil cytoplasmic antibodies, Goodpasture syndrome, acute rheumatic fever, myasthenia gravis, Graves disease, insulin-resistant diabetes, pernicious anemia, organ transplant rejection, blood-group incomparability resulting in hemolysis, paraneoplastic syndrome, systemic lupus erythematosus, psoriasis, poststreptococcal glomerulonephritis, acute glomerulonephritis, serum sickness, Arthus reaction, reactive arthritis, polyarteritis nodosa, contact dermatitis, multiple sclerosis, transplant rejection, rheumatoid arthritis, tuberculosis, peripheral neuropathy, or other immune diseases or disorders.

Examples of immunodeficiency disorders include, but are not limited to, X-linked agammaglobulinemia, common variable immunodeficiency, isolated IgA deficiency, hyper-IgM syndrome, DiGeorge syndrome, severe combined immunodeficiency disease (SCID), Wiskott-Aldrich syndrome, or a genetic deficiency of a complement system of the subject, Acquired Immunodeficiency Syndrome (AIDS), human immunodeficiency virus (HIV) infection, combined immune deficiency syndrome (CIDS), or a spinal cord injury-induced immune depression syndrome (SCI-IDS).

Effectiveness of energy delivery can be monitored by assessing peripheral blood samples, and energy dose adjustments may be made based on these bloodwork results, for example.

The one or more transducers described herein may be single use, disposable, or may be multi-use, and non-disposable. The non-disposable transducers may be sterilized to provide multiple uses.

The apparatuses described herein, and methods of using them, may also be used in any tissue in the body, including in particular, body regions having a natural body lumen, such as blood vessels, lymph vessels, lung cavities, and any other vessel, tube, tracts, canals, etc. within the body.

In some non-limiting embodiments described herein is a system including a control system that generates a frequency waveform and one or more transducers configured to deliver an ultrasound frequency waveform to a treatment area of a subject, wherein said treatment area comprises a lymphatic vessel, in one example. The delivery of the ultrasound frequency to the lymphatic vessel targets cytokines and white blood cells (WBCs) in some examples. The system may include an ultrasound energy which may be modulated such that it may be tuned to a single or a plurality of energies, frequencies and/or duty cycles dependent on the therapeutic target of the system. In one embodiment, the lymphatic vessel(s) nay include the left thoracic duct or the right thoracic duct, or a combination thereof. In one example, the ultrasound is targeted to the left or right thoracic duct, or both. The ultrasound frequency may be delivered continuously, or over a plurality of duty cycles. The delivery of the ultrasound frequency may cause apoptosis of the WBC's (white blood cells) in the treatment area, or downstream thereof. The treatment area may include the lymphatic vessel(s).

In one embodiment, the ultrasound frequency is delivered via one or more transducers. The one or more transducers may be configured to be positioned in or around the supraclavicular fossa, in one example, to access or target the treatment area. The one or more transducers, or other components of the system may be waterproof.

In one embodiment, a method is provided for suppressing an immune system of a subject. The method includes administering to a lymphatic vessel(s) of the subject an amount of directed ultrasound energy. Administering to the subject includes directing one or more beams of ultrasound energy to or at the lymphatic vessel(s) of the subject.

In another embodiment, a method of treating a disease in a subject is provided. The method includes delivering ultrasound energy to a treatment area of the subject. The ultrasound energy may be tuned to inactivate the cytokines in the treatment area or downstream thereof by breaking down their quaternary molecular structure.

In one embodiment, the treatment area comprises one or more lymphatic vessels of the subject. In another embodiment, the lymphatic vessel comprises a left or right thoracic duct. In yet another, non-limiting embodiment, there is provided a method of adjusting an ultrasound energy delivery to a thoracic duct of a subject based on peripheral blood samples which measure the levels, or changes in the levels of target cells, cytokines, or the activity thereof.

In yet another embodiment, there is provided a method of treating a disease in a subject. The method includes a system including a control system that generates a frequency waveform and one or more transducers configured to deliver an ultrasound frequency waveform to a treatment area of a subject, wherein said treatment area comprises a lymphatic vessel. The delivery of the ultrasound frequency to the lymphatic vessel targets cytokines and white blood cells (WBCs) in some examples. The system may include an ultrasound energy which may be modulated such that it may be tuned to a single or a plurality of energies, frequencies and/or duty cycles dependent on the therapeutic target of the system. In one embodiment, the lymphatic vessel(s) nay include the left thoracic duct or the right thoracic duct, or a combination thereof. In one example, the ultrasound is targeted to the left or right thoracic duct, or both. The ultrasound frequency may be delivered continuously, or over a plurality of duty cycles. The delivery of the ultrasound frequency may cause apoptosis of the WBC's (white blood cells) in the treatment area, or downstream thereof. The treatment area may include the lymphatic vessel(s). The method includes wherein the disease comprises an immune disorder selected from the group consisting of an autoimmune disorder, a hypersensitivity syndrome, an immune deficiency syndrome, and combinations thereof.

In still another embodiment, a method of treating an immune disease or disorder in a subject is provided. The method includes administering a dose of directed ultrasound to the thoracic duct(s) of the subject to inactivate certain pro-inflammatory cytokines in the subject and/or inactivate certain WBCs in the subject, to achieve negative modulation of the immune system.

In another embodiment, a method of ultrasound delivery to a thoracic duct comprising certain inactivated anti-inflammatory cytokines and/or inactivated WBC's, wherein the ultrasound delivery activates the cytokines and/or WBC's to achieve positive modulation of the immune system.

In yet another embodiment, the ultrasound frequency waveform comprises between 50 to 200 W/cm2 spatial peak temporal average acoustic output intensity (Ispta). In another example, the ultrasound may be delivered at a frequency between 75 and 100 W/cm2 spatial peak temporal average acoustic output intensity (Ispta). In still another embodiment, the ultrasound frequency may be delivered for a duration of 0.5 seconds to 1 minute per duty cycle. In yet another embodiment, the ultrasound frequency may be delivered for a duration of 1 minute to 5 minutes per duty cycle.

Turning to the Figures, FIG. 1 includes an embodiment of an ultrasound transducer 100 including a transducer body 110 and a transducer cable 112. The transducer body 110 may include a combination of an ultrasound conducting component 114, which in one example may include viscoelastic ultrasound conducting material, ultrasound reflective component 116, a piezoelectric component or layer 118, and a gas sterilizable component 120, for example. The ultrasound transducer may include any combination of these components in other non-limiting embodiments. For example, an ultrasound transducer as described herein may include one or more of these components. In another embodiment an ultrasound transducer as described herein may include two or more of these components. The piezoelectric component 118 may be used to generate ultrasound waves, in one embodiment. The piezoelectric component 118 may also be used for receiving an ultrasound wave. In another embodiment, the piezoelectric component 118 may be used to receive and transmit an ultrasound wave.

The ultrasound transducer 100 may be connected to a transmitting unit which supplies a transmission ultrasound signal to the ultrasound transducer; a receiving unit which performs predetermined signal processing on a reception signal received by the ultrasound transducer; and optionally, an image processing unit which creates a tomographic image of an internal state of the subject based on the reception signal from the receiving unit. The ultrasound transducer may include a number of electrodes arranged at interlayers and/or on surfaces of the layers of the ultrasound transducer body layers described above, in one embodiment.

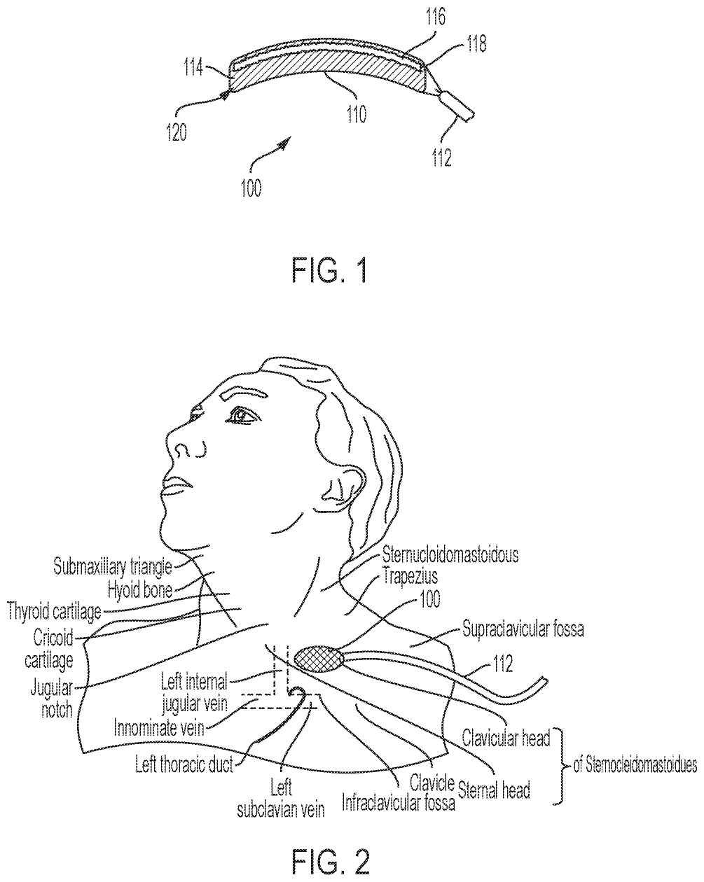

FIG. 2 provides a schematic of the anatomy of the head and neck region of a subject and the ultrasound transducer embodiment 100 superimposed thereon. As described above, the one or more ultrasound transducer(s) 100 may be placed at, on, near, or within the lymphatic vessel of a subject, for example, the thoracic duct(s) of the subject, to deliver ultrasonic waves to the vessel. FIG. 2 shows an embodiment of the ultrasound transducer 100 positioned at or near the supraclavicular fossa such that its energy is directed toward a vessel, for example, toward the left thoracic duct, for treatment of the subject to positively or negatively modulate the immune system by delivering ultrasound waves to the vessel.

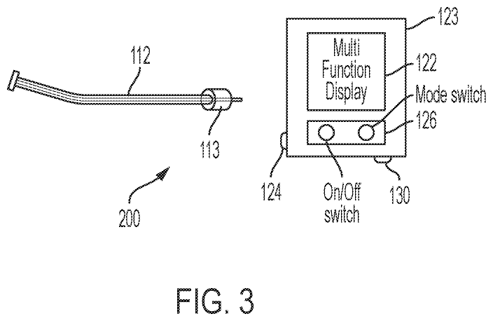

FIG. 3 is a schematic showing a system 200 including a transducer cable 112 for connecting to a transducer 100 (not shown in FIG. 3), and a plug 113 for connecting the transducer cable 112 to a control system 123 including a multifunction display 122 and a user input panel 126 which may include a number of responsive members including digital input members, switches, or physical compressible members, for example an on/off component for turning the system on or off, a mode component, which may include a switch for changing the mode of operation of the system or the mode on the multi-function display 122, and a number of other components accessible and adjustable via the user input panel 126. The system may further include a power or charging receptacle 130 and a transducer receptacle 128 for receiving the connection to the transducer cable 112 to connect the ultrasound transducer 100 for operating and, optionally, providing power to the ultrasound transducer 100 in one embodiment.

In one embodiment described herein is a method of suppressing an immune system of a subject, including administering to a lymphatic vessel (or vessels) of the subject an amount of directed ultrasound energy. In one embodiment, the lymphatic vessel may include the thoracic duct(s).

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

The term “associated” or “association”, as used herein, includes but is not limited to direct and indirect attachment, adjacent to, in contact with, partially or fully attached to, and/or in close proximity therewith. The term “in conjunction with” as used herein includes but is not limited to synchronously or near synchronous timing, the phrase may also include the timing of outputs, where one output directly follows another output.

As used herein, the terms “subject” , “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

What is claimed is:

1. A system, comprising:
a control system that generates a frequency waveform; and
one or more transducers configured to deliver an ultrasound frequency waveform to a treatment area of a subject, wherein said treatment area comprises a lymphatic vessel;
wherein delivery of the ultrasound frequency to the lymphatic vessel targets cytokines and white blood cells (WBCs).

2. The system of claim 1, wherein the ultrasound energy may be tuned to one, or a plurality of energies, frequencies, and duty cycles, depending on the therapeutic target.

3. The system of claim 1, wherein the lymphatic vessel comprises the left thoracic duct or the right thoracic duct, or a combination thereof.

4. The system of claim 1, wherein the ultrasound frequency is delivered either continuously or over a plurality of duty cycles.

5. The system of claim 4, wherein the ultrasound frequency is delivered for a duration of 0.5 seconds to 1 minute per duty cycle.

6. The system of claim 1, wherein delivery of the ultrasound frequency causes apoptosis of WBCs in the treatment area or downstream thereof.

7. The system of claim 1, wherein the one or more transducers are configured for positioning in or around the supraclavicular fossa.

8. The system of claim 1, wherein the one or more transducers are waterproof.

9. A method of treating a disease in a subject, comprising:
delivering ultrasound energy to a treatment area of the subject, wherein said ultrasound energy inactivates the cytokines in the treatment area or downstream thereof.

10. The method of claim 9, wherein the ultrasound energy inactivates the cytokines by disrupting the molecular structure of the cytokines.

11. The method of claim 9, wherein the treatment area comprises a lymphatic vessel of the subject.

12. The method of claim 11, wherein the lymphatic vessel comprises a thoracic duct.

13. The method of claim 9, wherein the ultrasound energy delivered is adjusted based on peripheral blood samples which measure the levels, or changes in the levels of target cells, cytokines, or the activity thereof.

14. A method of treating a disease in a subject comprising the system of claim 1, wherein the disease comprises an immune disorder selected from the group consisting of an autoimmune disorder, a hypersensitivity syndrome, an immune deficiency syndrome, and combinations thereof; and the method comprises:
administering a dose of directed ultrasound to the thoracic duct(s) of the subject to inactivate pro-inflammatory cytokines in the subject and/or inactivate WBCs in the subject, to achieve negative modulation of the immune system.
